(19) United States
(12) Patent Application Publication
(10) Pub. No.: US 2024/0009397 A1
(43) Pub. Date: Jan. 11, 2024

(54) SYSTEMS AND METHODS FOR ROTATIONAL PIERCING OF PRE-FILLED MEDICAL DELIVERY ASSEMBLIES

(71) Applicant: Koska Family Limited, East Sussex (GB)

(72) Inventors: Hanjin In, Toronto (CA); Marc Andrew Koska, Dartmouth (GB); Jeff Price, Windermere, FL (US)

(21) Appl. No.: 18/265,229

(22) PCT Filed: Dec. 6, 2021
(86) PCT No.: PCT/US21/61991

§ 371 Date: Jun. 2, 2023

Related U.S. Application Data
(60) Provisional application No. 63/121,411, filed on Dec. 4, 2020.

(51) Int. Cl. A61M 5/24

(52) U.S. Cl. CPC A61M 5/2466; A61M 5/2425; A61M 2005/2474; A61M 2205/197; A61M 2205/195

(57) ABSTRACT

A pre-filled medical delivery assembly assembled and configured to allow delivery of a single dose of a therapeutic agent (e.g., vaccine, drug, medicament, etc.) from a Blow-Fill-Seal (BFS) vial to a patient may include a multi-bevel cannula piercing element such as for rotational axial engagement to pierce a seal of the BFS vial.

### CROSS-REFERENCE TO RELATED APPLICATIONS

Benefit and priority is claimed to, and this is a National Stage pursuant to 35 U.S.C. § 371 of, International Patent Application PCT/US21/061991 filed on Dec. 6, 2021 and titled “SYSTEMS AND METHODS FOR ROTATIONAL PIERCING OF PRE-FILLED MEDICAL DELIVERY ASSEMBLIES”, which itself claims benefit and priority under 35 U.S.C. § 119(e) to, and is a Non-provisional of, U.S. Provisional Patent Application No. 63/121,411 filed on Dec. 4, 2020 and titled “MULTI-BEVEL CANNULA”. Each of these Applications is hereby incorporated by reference herein in its entirety and for all purposes.

### BACKGROUND

Every year, millions of people become infected and die from a variety of diseases, some of which are vaccine-preventable. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs generally include administration of vaccines via a typical reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting the death of more than one (1) million people each year.

Previous attempts at providing single-use or disposable injection devices to remedy such problems in the industry have achieved some success but have failed to adequately remedy the existing problems. Pre-filled, single-use injection devices manufactured via injection molding or Form-Fill-Seal (FFS) processes, such as the Uniject™ device (which is an example of an activated or pre-activated device, as the cannula is embedded to be in communication with the pre-filled contents during the manufacturing process) available from the Becton, Dickinson and Company of Franklin Lakes, NJ, for example, while offering precise manufacturing tolerances in the range of two thousandths of an inch (0.002-in; 50.8 μm) to four thousandths of an inch (0.004-in; 101.6 μm) —for hole diameters in molded parts, require separate sterilization processes (e.g., gamma radiation) that are not compatible with certain fluids, provide production rates limited to approximately nine thousand (9,000) non-sterile units per hour, and can be provided to an end-user for approximately one dollar and forty cents ($1.40) per dose/unit.

### BRIEF DESCRIPTION OF THE DRAWINGS

An understanding of embodiments described herein and many of the attendant advantages thereof may be readily obtained by reference to the following detailed description when considered with the accompanying drawings, wherein:

FIG. 1A, FIG. 1B, FIG. 10, FIG. 1D, FIG. 1E, and FIG. 1F are various views of a pre-filled medical delivery assembly according to some embodiments;

FIG. 2A, FIG. 2B, FIG. 2C, FIG. 2D, FIG. 2E, FIG. 2F, and FIG. 2G are various views of a pre-filled medical delivery assembly coupling according to some embodiments;

FIG. 3A, FIG. 3B, FIG. 3C, FIG. 3D, and FIG. 3E are various views of a multi-bevel cannula according to some embodiments; and

FIG. 4 is a partial perspective view of a multi-bevel cannula pre-filled medical delivery system according to some embodiments.

### DETAILED DESCRIPTION

### I. Introduction

Embodiments of the present invention provide systems and methods for multi-bevel cannulas that overcome drawbacks of non-activated pre-filled medical delivery assemblies. Such non-activated pre-filled medical delivery assemblies may comprise, for example, a Blow-Fill-Seal (BFS) vial or bottle coupled to a specialized collar that facilitates coupling of an administration member (e.g., a needle or cannula) to the BFS vial. In some embodiments, such a non-activated pre-filled medical delivery assembly may be selectively actuated by application of rotational force to a cap covering the administration member, causing the administration member to axially advance and pierce a fluid reservoir of the BFS vial. In some embodiments, such as in the case that the administration member comprises a cannula, the cannula may comprise two pointed tips, one at each end—e.g., one for piercing the BFS vial and one for injection into a patient. According to some embodiments, the engagement of the cannula with the BFS vial may comprise an advancement of one of the pointed tips into the BFS vial (e.g., to pierce a seal or wall thereof).

Utilization of such systems that employ BFS vials may be advantageous and may address various shortcomings of previous systems. BFS vials may, for example, offer a less expensive alternative to typical vials or devices created via other manufacturing techniques. In some embodiments, BFS vials (e.g., due to the nature of the BFS manufacturing process) may not require separate sterilization (e.g., and may accordingly be compatible with a wider array of fluids), may provide enhanced production rates of sterile/aseptic units per hour, and/or may be provided to an end-user for significantly lower per dose/unit costs. In some embodiments, these advantages may come with an attendant drawbacks of reduced manufacturing tolerances and other disadvantages of utilizing a “soft” plastic (e.g., having a Shore/Durometer “D” hardness of between 60 and 70). BFS processes may, for example, offer less precise manufacturing tolerances in the range of five hundredths of an inch (0.05-in; 1.27 mm) to fifteen hundredths of an inch (0.15-in; 3.81 mm)—for linear dimensions, e.g., in accordance with the standard ISO 2768-1 “General tolerances for linear and angular dimensions without individual tolerance indications” published by the International Organization for Standardization (ISO) of Geneva, Switzerland (Nov. 15, 1989) and/or may not be readily adaptable to form certain mating features such as standardized threads.

According to some embodiments, piercing of the non-activated pre-filled medical delivery assemblies that utilize pre-filled BFS vials may present difficulties due to the nature of the piercing element and the “soft” plastic of the BFS vial. Attempts to pierce BFS vials in a rotational manner utilizing a standard pointed needle/cannula tip, for example, have shown a tendency of the BFS vial (or the seal/wall thereof) to be “cored” by a rotating needle/cannula tip. “Coring” is utilized herein to describe a separation of a portion of the BFS vial due to engagement of the needle/cannula. Applicant has observed that various configurations of needle/cannula tips may cause coring during rotational engagement with a BFS vial/container, which may cause a separated portion of the BFS vial/container to become lodged inside the needle/cannula, fall into the pre-filled fluid, and/or advance with the pre-filled liquid into an administration target. Any of these coring effects are highly undesirable. In some embodiments, these drawbacks and/or the deficiencies of prior systems may be advantageously addressed by specific features, configurations, and/or components as described hereinafter.

### II. Pre-Filled Medical Delivery Assemblies—Exterior Threaded Coupling

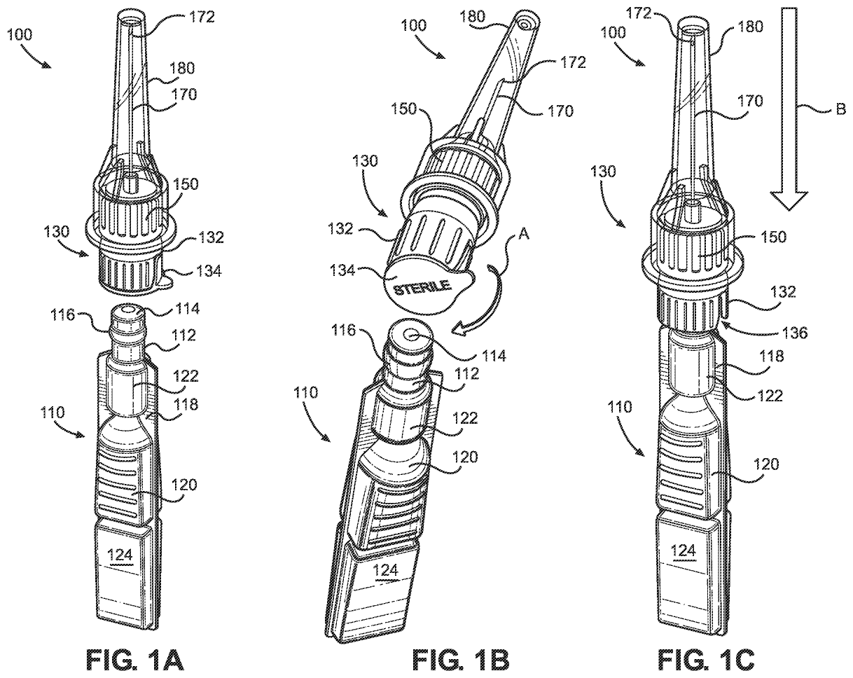

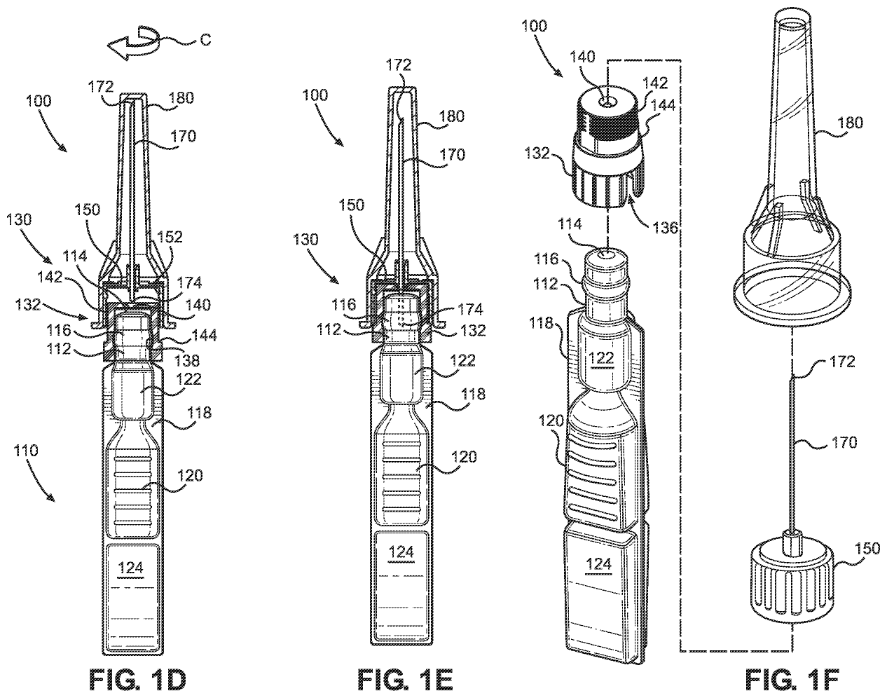

Referring initially to FIG. 1A, FIG. 1B, FIG. 1C, FIG. 1D, FIG. 1E, and FIG. 1F, various views of a pre-filled medical delivery assembly 100 according to some embodiments are shown. In some embodiments, the pre-filled medical delivery assembly 100 may comprise various inter-connected and/or modular components such as a BFS vial 110 comprising and/or defining a vial neck 112, a fluid seal 114, a mounting flange 116, a bottle flange 118, a collapsible reservoir 120, a dispensing reservoir 122, and/or an identification area 124. According to some embodiments, the pre-filled medical delivery assembly 100 may comprise an administration (e.g., injection) module or component 130 that is manufactured, assembled, and/or provided as a separate unit from the BFS vial 110. In some embodiments, the administration component 130 may comprise a mounting collar 132 which itself comprises, is coupled to, and/or defines various features and/or elements. The mounting collar 132 and/or the administration assembly 130 may, for example, be maintained as a closed and/or sterile component via a seal 134 (e.g., a foil, wax, paper, and/or other thin, pierceable, tear-able, and/or removable object or layer coupled to the mounting collar 132 and/or the administration component 130) that seals an interior volume or socket (not separately labeled) of the mounting collar 132, disposed at a first end thereof. According to some embodiments, the mounting collar 132 may comprise one or more coupling or mounting features 136, an internal seat 138 (e.g., that is configured to accept the mounting flange 116 of the BFS vial 110 in the case that the neck 112 of the BFS vial 110 is inserted into the mounting collar 132 and/or the administration component 130), a puncture seal 140, and/or external threads 142. In some embodiments, the mounting collar 132 may comprise and/or define an exterior flange 144 (e.g., a radial flange) that is operable to receive, mate with, and/or otherwise engage with a needle hub 150. In some embodiments, the needle hub 150 may couple to the mounting collar 132 via threads 152 thereof that correspond to and/or mate with the threads 142 of the mounting collar 132. According to some embodiments, the needle hub 150 may comprise, couple to and/or house a needle, cannula, and/or other administration member 170 (such as a multi-bevel, dual-tipped cannula as described herein), and/or a cap 180 (e.g., selectively engaged and/or coupled to the needle hub 150 to shroud, house, and/or protect the administration member 170). According to some embodiments, the pre-filled medical delivery assembly 100 may include a modular design consisting of separately constructed components 110, 130, 132, 150, 170, 180 cooperatively arranged and coupled to one another.

In some embodiments, the collapsible reservoir 120 may be filled (fully or partially) with a fluid or other agent (not separately shown) to be delivered, e.g., to a patient (not shown). According to some embodiments, the fluid may be injected into the BFS vial 110 in a sterile environment during manufacture via a BFS process and sealed within the BFS vial 110 via the fluid seal 114. The fluid seal 114 may comprise a portion of the molded BFS vial 110 for example that is configured to be pierced to expel the fluid, e.g., such as by providing a flat or planar piercing surface and/or by being oriented normal to an axis of the BFS vial 110 (and/or the pre-filled medical delivery assembly 100). In some embodiments, the fluid seal 114 may comprise a foil, wax, paper, and/or other thin, pierceable object or layer coupled to the BFS vial 110. In some embodiments, the neck 112 of the BFS vial 110 may comprise the mounting flange 116 such as, e.g., the “doughnut”-shaped exterior flange depicted. The mounting flange 116 may, for example, provide a radially elastic mating surface that is operable to provide a selective engagement or fit within the socket of the administration component 130.

According to some embodiments, the fluid may generally pass between the collapsible reservoir 120 and the connected dispensing reservoir 122. In some embodiments, a juncture, valve, and/or passage (not separately labeled) between the dispensing reservoir 122 and the collapsible reservoir 120 may restrict flow such that the fluid may readily enter one of the dispensing reservoir 122 and the collapsible reservoir 120 but may not readily return to the other reservoir 120, 122. In some embodiments, the constriction may not be necessary or desirable, such as in the case that the collapsible reservoir 120 and the dispensing reservoir 122 are formed and/or combined as a single, unobstructed reservoir, e.g., a single fluid reservoir (not shown).

In some embodiments, the pre-filled medical delivery assembly 100 may include a modular design consisting of separately constructed components 110, 130 cooperatively arranged and coupled to one another. As depicted in FIG. 1A, for example, the BFS vial 110 and the administration component 130 may be manufactured, packaged, shipped, stored, and/or provided as separate components. In such a manner, the administration component 130 may not need to be stored or shipped in accordance with often restrictive requirements imposed on medicaments and may accordingly reduce the amount of space required for such specialized storage and/or shipping. The administration component 130 may also or alternatively be manufactured, stored, and/or shipped in advance (e.g., at a first time) while the BFS vial 110 that is pre-filled with the fluid may be manufactured, stored, and/or shipped at a later time (e.g., a second time). In some embodiments, the delay between the first time and the second time may be lengthy without causing determinantal effects, as the administration component 130 may be stored, in some embodiments, indefinitely. In such a manner, units of the administration component 130 may be provided to be on-hand in advance of the availability and/or arrival of the BFS vial 110, reducing supply chain constraints in the case of proactive administration component 130 procurement.

According to some embodiments, the components 110, 130 may be coupled, e.g., in the field and/or in situ, to provide an active pre-filled (e.g., injectable) medical delivery device. As shown in FIG. 1B, for example, the seal 134 may be removed from the administration component 130 (at “A”) and the administration component 130 (and/or the socket thereof) may be aligned with the neck 112 of the BFS vial 110. According to some embodiments, the administration component 130 (and/or the mounting collar 132 thereof) may be axially engaged to couple with the BFS vial 110 via application of a mating axial force, as shown in FIG. 10 (at “B”). The administration component 130 (and/or the mounting collar 132 thereof) may be urged onto the neck 112 of the BFS vial 110, for example, such that the cooperatively shaped internal seat 138 (e.g., an interior groove or channel) accepts the mounting flange 116, thereby removably coupling the BFS vial 110 and the administration component 130 (and/or the mounting collar 132 thereof). In some embodiments, the internal seat 138 (and/or other interior features) and/or the mounting flange 116 may be shaped such that uncoupling of the BFS vial 110 and the administration component 130 (and/or the mounting collar 132 thereof) is mechanically prohibited. In some embodiments, the mounting flange 116 may be shaped as an axially elongated rounded exterior flange (e.g., the “doughnut” shape as depicted) and/or the internal seat 138 may comprise a cooperative and/or mirrored axially elongated rounded interior groove or track. According to some embodiments, the one or more mounting features 136 such as the mirrored axial slits depicted may engage with the bottle flange 118 (and/or portions of the BFS vial 110) such that rotation of the administration component 130 (and/or the mounting collar 132 thereof) with respect to the BFS vial 110 is restricted in the case that they are coupled. In some embodiments, the coupling of the BFS vial 110 and the administration component 130 (and/or the mounting collar 132 thereof) may be configured to explicitly permit free rotation (e.g., about a common axis) of the BFS vial 110 with respect to the administration component 130 (and/or the mounting collar 132 thereof). The mounting features 136 may not, in some embodiments for example, engage with the BFS vial 110 (and/or the mounting flange 116 thereof), e.g., to permit rotation therebetween.

As depicted in FIG. 1D, in some embodiments the neck 112 of the BFS vial 110 may be urged and/or forced into the socket of the mounting collar 132 until the mounting flange 116 becomes seated in (and/or coupled to or mated with) the internal seat 138 (e.g., a seated position). In such a manner, the fluid seal 114 may be advantageously positioned adjacent to the puncture seal 140. According to some embodiments, the mounting flange 116 may be configured as the doughnut shape (as depicted) to provide various advantages to the pre-filled medical delivery assembly 100. The axial elongation of the mounting flange 116 may, for example, provide for a smooth, uniform, and/or less forceful mating process that is less likely to deform the soft plastic neck 112 of the BFS vial 110 and/or may provide for a lengthened mating surface that is more likely to prevent leakage of the fluid. In some embodiments, the mounting flange 116 and the cooperatively shaped and sized internal seat 138 may permit simple, effective, and/or economic attachment of the administration component 130 to the BFS vial 110.

In some embodiments, the needle hub 150 may be coupled to the mounting collar 132 via engagement of the external threads 142 of the mounting collar 132 with the internal threads 152 of the needle hub 150. The internal threads 152 that correspond and cooperate with the external threads 142 such that they may be rotationally and/or removably coupled. According to some embodiments, the administration component 130 may be provided with the mounting collar 132 and the needle hub 150 partially engaged (e.g., with the threads 142, 152 being partially coupled), such as depicted in FIG. 1D. In some embodiments, the partial engagement (or first engagement state) may cause the administration member 170 (e.g., a second or proximal end thereof) to be positioned adjacent to (and/or in contact with) the puncture seal 140 of the mounting collar 132. In such a first engagement state, the administration component 130 may be coupled to the BFS vial 110, but the BFS vial 110 (and/or the puncture seal 140) have not yet been punctured and/or breached by the administration member 170. In other words, the pre-filled medical delivery assembly 100 in FIG. 1A, FIG. 1B, FIG. 10, and FIG. 1D is in a non-activated or pre-activated state.

According to some embodiments the needle hub 150 may couple to and/or retain the administration member 170. The administration member 170 may be inserted into and/or through the needle hub 150, for example, such that it comprises a first or administration end 172 extending axially distal from the BFS vial 110 and a second or piercing end 174 disposed within the needle hub 150. In some embodiments, the administration end 172 and/or a distal portion of the administration member 170 may be housed, shrouded, and/or covered by the cap 180. According to some embodiments, the cap 180 may be configured to house the administration member 170 and to removably couple to the housing 150 (e.g., by fitting over an external portion thereof and/or by engaging with the external flange 144).

According to some embodiments, the mounting collar 132 and needle hub 150 combination may be utilized to couple and/or mate the administration member 170 with the BFS vial 110 to provide a mechanism via which the administration member 170 may be coupled to the soft plastic BFS vial 110 in a reliable manner. Due to the nature of the BFS plastic and/or process and/or the small form-factor of the BFS vial 110, for example, providing standard external threads (not shown) directly on the neck 112 would not be a viable option for it would result in an imprecise, unreliable, and/or non-water tight coupling (i.e., the threads would be deformable even if they could be properly manufactured to within the desired tolerances, which itself is not a likely result) between he BFS vial 110 and, e.g., the needle hub 150. Applicant has realized, for example, that “soft” plastics required for the BFS process are not susceptible to machining due to heat deformation of machined features during formation attempts as well as deformation due to mechanical stress during utilization. As such, standardized screw-on needle hubs (not shown; although similar to the needle hub 150, in some embodiments) are not readily compatible for attachment directly to BFS vials 110 (e.g., in the absence of the mounting collar 132).

In some embodiments, the administration member 170 may include a needle shaped and/or sized for at least one of subcutaneous, intramuscular, intradermal, and intravenous injection of the fluid agent into a patient (not shown). For ease of explanation and description, the figures and the description herein generally refer to the administration member 170 as a needle or cannula. However, it should be noted that, in other embodiments, the administration member 170 may include (e.g., at the administration end 172 thereof) a nozzle (not shown) configured to control administration of the fluid agent to the patient. The nozzle may include a spray nozzle, for example, configured to facilitate dispersion of the fluid agent into a spray. Accordingly, a needle hub 150 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a needle hub 150 including a droplet nozzle may be useful in the administration of a fluid agent by way of droplets, such as administration to the eyes, topical administration, and the like.

As generally understood, the fluid or drug (e.g., stored in the BFS vial 110 and/or one or more of the reservoirs 120, 122 thereof) agent may include any type of agent to be injected into a patient (e.g., animal such as a mammal, either human or non-human) and capable of producing an effect (alone, or in combination with an active ingredient). Accordingly, the agent may include, but is not limited to, a vaccine, a drug, a therapeutic agent, a medicament, a diluent, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the BFS vial 110 (e.g., at, on, or in the identification area 124) and/or the administration component 130.

According to some embodiments, the mounting collar 132, the needle hub 150, and/or the cap 180 may be composed of a medical grade material. In some embodiments, the mounting collar 132, the needle hub 150, and/or the cap 180, may be composed of a thermoplastic polymer or other “hard” plastic (e.g., greater than 80 on the Rockwell “R” scale), including, but not limited to, polybenzimidazole, acrylonitrile butadiene styrene (ABS), polystyrene, polyvinyl chloride, or the like. In some embodiments, the pre-filled medical delivery assembly 100 may be advantageously manufactured (in mass quantities) in separate parts or portions, namely, at least the “soft” plastic BFS vial 110 portion (e.g., a “first” piece) and the “hard” plastic administration component 130 (e.g., the “second” piece), with such different plastic parts/portions being selectively coupled to administer a medication to a patient.

According to some embodiments, the pre-filled medical delivery assembly 100 may be advanced from the first engagement state (i.e., the non-activated state) to a second engagement (or activation) state where the administration member 170 has pierced the BFS vial 110 and the fluid therein may readily be expressed through the administration member 170 (e.g., and into a patient). The partial engagement of the mounting collar 132 and the needle hub 150 (e.g., with the threads 142, 152 being partially coupled) as depicted in FIG. 1D may, for example, be transitioned to a more advanced, fully, or completely advanced state by application of a rotational force to the cap 180, e.g., as shown in FIG. 1D (at “C”). In some embodiments, the more advanced and/or full engagement (or second engagement state; as shown in FIG. 1E) may cause the administration member 170 (e.g., the second or proximal end 174 thereof) to advance through the puncture seal 140 of the mounting collar 132 and through the seal 114 of the BFS vial 110. In such a manner, for example, the continued rotational engagement of the threads 142, 152 may cause the administration member 170 (e.g., the second or proximal end 174 thereof) to open a fluid pathway between the BFS vial 110 (e.g., and/or the neck 112, and/or reservoirs 120, 122 thereof) and the first or distal end 172 of the administration member 170. In some embodiments, the cap 180 may be utilized as a rotational force driver to transfer rotational force to the needle hub 150 to cause the advancement of the engagement of the threads 142, 152 (and, e.g., the puncturing of the puncture seal 140 and the seal 114 of the BFS vial 110).

According to some embodiments, the second or piercing end 174 of the administration member 170 may comprise a multi-bevel tip as described herein. The multi-bevel tip may, for example, decrease the likelihood of coring of either the puncture seal 140 and the seal 114, thereby reducing the likelihood of contamination of the fluid agent and/or blockage of the administration member 170. While standard tips may function adequately in the case of a linear axial piercing motion (not shown herein), for example, the rotational axial advancement of the second or piercing end 174 of the administration member 170 in embodiments described herein may produce undesirable results in the case that a standard or simple tip is utilized on the second or piercing end 174 of the administration member 170. In some embodiments, the first or distal end 172 of the administration member 170 may comprise any type or configuration of tip that is or becomes known or practicable for engagement with an administration target (e.g., a patient). According to some embodiments, the tip of the first or distal end 172 of the administration member 170 may comprise a multi-bevel tip similar to the second or piercing end 174 of the administration member 170 (e.g., such that during manufacturing and/or assembly of the administration component 130 the administration member 170 may be oriented in either longitudinal manner without change in effect).

In some embodiments, fewer or more components 110, 112, 114, 116, 118, 120, 122, 124, 130, 132, 134, 136, 138, 140, 142, 144, 150, 152, 170, 172, 174, 180 and/or various configurations of the depicted components 110, 112, 114, 116, 118, 120, 122, 124, 130, 132, 134, 136, 138, 140, 142, 144, 150, 152, 170, 172, 174, 180 may be included in the pre-filled medical delivery assembly 100 without deviating from the scope of embodiments described herein. In some embodiments, the components 110, 112, 114, 116, 118, 120, 122, 124, 130, 132, 134, 136, 138, 140, 142, 144, 150, 152, 170, 172, 174, 180 may be similar in configuration and/or functionality to similarly named and/or numbered components as described herein. According to some embodiments, the pre-filled medical delivery assembly 100 may comprise the mounting flange 116 but not the collapsible reservoir 120. In some embodiments, the pre-filled medical delivery assembly 100 may comprise the mounting flange 116 but not the dispensing reservoir 122. According to some embodiments, the administration component 130 may be provided without (and/or separately from) the BFS vial 110. According to some embodiments, the puncture seal 140 may instead comprise an opening or orifice or may not be included in the mounting collar 132. In some embodiments, the administration member 170 may be provided separate from the BFS vial 110 and/or the administration component 130.

### III. Pre-Filled Medical Delivery Assemblies—Interior Threaded Coupling and Safety Shield

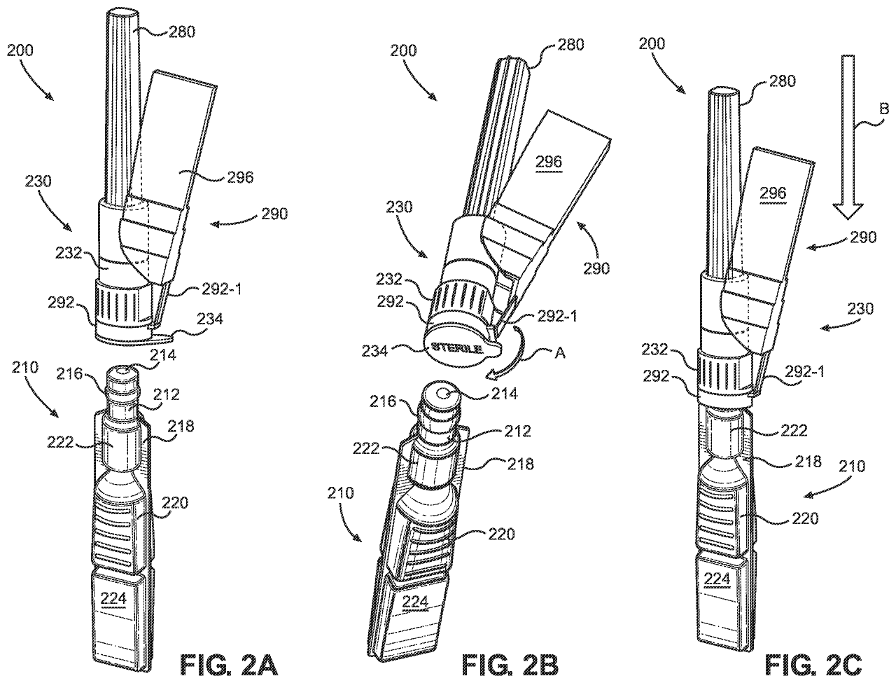

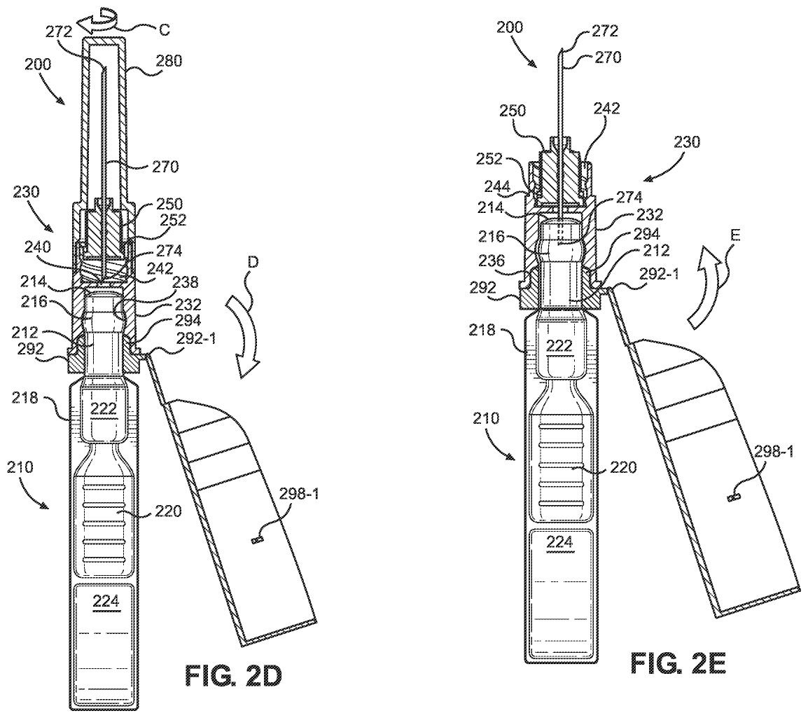

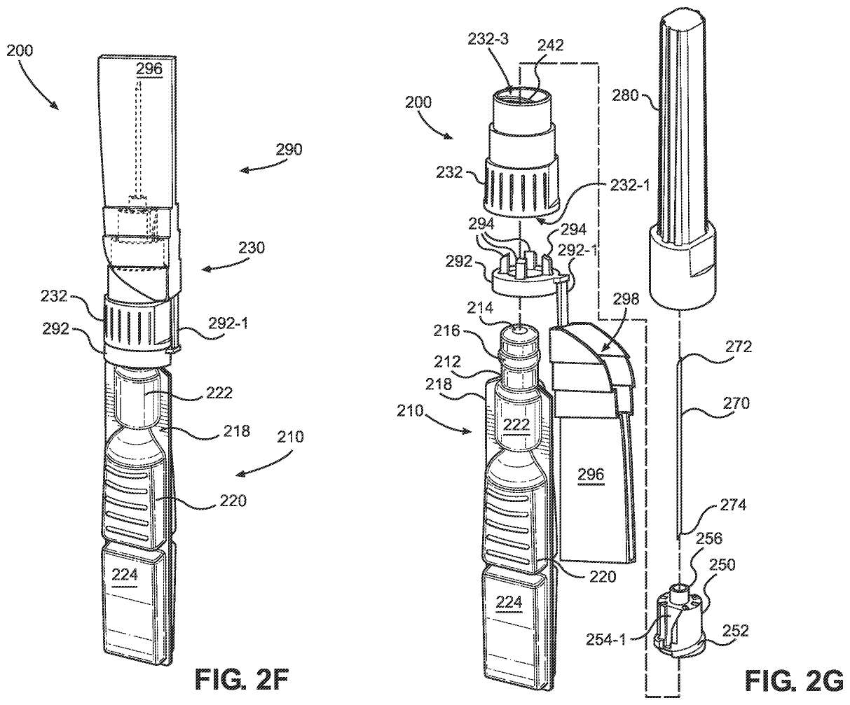

Turning to FIG. 2A, FIG. 2B, FIG. 2C, FIG. 2D, FIG. 2E, FIG. 2F, and FIG. 2G, various views of a pre-filled medical delivery assembly 200 according to some embodiments are shown. In some embodiments, the pre-filled medical delivery assembly 200 may comprise various inter-connected and/or modular components such as a BFS vial 210 comprising and/or defining a vial neck 212, a fluid seal 214, a mounting flange 216, a bottle flange 218, a collapsible reservoir 220, a dispensing reservoir 222, and/or an identification area 224. According to some embodiments, the pre-filled medical delivery assembly 200 may comprise an administration (e.g., injection) module or component 230 that is manufactured, assembled, and/or provided as a separate unit from the BFS vial 210. In some embodiments, the administration component 230 may comprise a mounting collar 232 which itself comprises, is coupled to, and/or defines various features and/or elements. The mounting collar 232 and/or the administration assembly 230 may, for example, be maintained as a closed and/or sterile component via a seal 234 (e.g., a foil, wax, paper, and/or other thin, pierceable, tear-able, and/or removable object or layer coupled to the mounting collar 232 and/or the administration component 230) that seals an interior volume or socket 232-1 (labeled in FIG. 2G) of the mounting collar 232, disposed at a first end thereof. In some embodiments, the mounting collar 232 may comprise and/or define a needle hub socket 232-3 disposed at a second end thereof. According to some embodiments, the mounting collar 232 may comprise one or more coupling or mounting features 236 (e.g., internal axial grooves and/or seats), an internal (e.g., radial) flange or seat 238 (e.g., that is configured to accept the mounting flange 216 of the BFS vial 210 in the case that the neck 212 of the BFS vial 210 is inserted into the mounting collar 232 and/or the administration component 230), a puncture seal 240, and/or internal threads 242 (e.g., disposed and/or formed within the needle hub socket 232-3). In some embodiments, the mounting collar 232 may comprise and/or define an exterior flange 244 (e.g., a radial flange). According to some embodiments, a needle hub 250 may couple to the mounting collar 232 via external threads 252 thereof that correspond to and/or mate with the internal threads 242 of the mounting collar 232. In some embodiments, the needle hub 250 may comprise an external groove, projection, and/or drive surface 254-1 that may be rotationally engaged to manipulate the coupling of the threads 242, 252. According to some embodiments, the needle hub 250 (and/or a riser 256 thereof) may comprise, couple to and/or house a needle, cannula, and/or other administration member 270 (such as a multi-bevel, dual-tipped cannula as described herein; e.g., having a first or administration end 272 and a second or piercing end 274), and/or a cap 280 (e.g., selectively engaged and/or coupled to the mounting collar 232 (and/or the exterior flange 244 thereof) and/or the needle hub 250, e.g. to shroud, house, and/or protect the administration member 270).

In some embodiments, the administration component 230 may comprise and/or be coupled to a safety shield 290. The safety shield 290 may comprise, for example, a shield base 292 that is mounted on and/or around the neck 212 of the BFS vial 210. According to some embodiments, the shield base 292 may comprise and/or be coupled to a hinge element 292-1 and/or may comprise one or more mounting features 294. The hinge element 292-1 may flexibly couple the shield base 292 to a shield element 296, for example, the shield element 296 comprising a molded and/or shaped element configured to selectively cover the administration member 270. In some embodiments, the shield element 296 may comprise and/or define a shield volume or space 298 that is sized and/or shaped to accept and/or house the administration member 270. In some embodiments, the shield space 298 may comprise, define, and/or house a needle keeper 298-1, e.g., in the case that the administration member 270 comprises a needle and/or cannula. According to some embodiments, the pre-filled medical delivery assembly 200 may include a modular design consisting of separately constructed components 210, 230, 232, 250, 270, 280, 290 cooperatively arranged and coupled to one another.

In some embodiments, the collapsible reservoir 220 may be filled (fully or partially) with a fluid or other agent (not separately shown) to be delivered, e.g., to a patient (not shown). According to some embodiments, the fluid may be injected into the BFS vial 210 in a sterile environment during manufacture via a BFS process and sealed within the BFS vial 210 via the fluid seal 214. The fluid seal 214 may comprise a portion of the molded BFS vial 210 for example that is configured to be pierced to expel the fluid, e.g., such as by providing a flat or planar piercing surface and/or by being oriented normal to an axis of the BFS vial 210 (and/or the pre-filled medical delivery assembly 200). In some embodiments, the fluid seal 214 may comprise a foil, wax, paper, and/or other thin, pierceable object or layer coupled to the BFS vial 210. In some embodiments, the neck 212 of the BFS vial 210 may comprise the mounting flange 216 such as, e.g., the “doughnut”-shaped exterior flange depicted. The mounting flange 216 may, for example, provide a radially elastic mating surface that is operable to provide a selective engagement or fit within the socket 232-1 of the administration component 230.

According to some embodiments, the fluid may generally pass between the collapsible reservoir 220 and the connected dispensing reservoir 222. In some embodiments, a juncture, valve, and/or passage (not separately labeled) between the dispensing reservoir 222 and the collapsible reservoir 220 may restrict flow such that the fluid may readily enter one of the dispensing reservoir 222 and the collapsible reservoir 220 but may not readily return to the other reservoir 220, 222. Such a constriction may in some embodiments, provided advantages as described herein. In some embodiments, the constriction may not be necessary or desirable, such as in the case that the collapsible reservoir 220 and the dispensing reservoir 222 are formed and/or combined as a single, unobstructed reservoir, e.g., a single fluid reservoir (not shown).

In some embodiments, the pre-filled medical delivery assembly 200 may include a modular design consisting of separately constructed components 210, 230 cooperatively arranged and coupled to one another. As depicted in FIG. 2A, for example, the BFS vial 210 and the administration component 230 may be manufactured, packaged, shipped, stored, and/or provided as separate components. In such a manner, the administration component 230 may not need to be stored or shipped in accordance with often restrictive requirements imposed on medicaments and may accordingly reduce the amount of space required for such specialized storage and/or shipping. The administration component 230 may also or alternatively be manufactured, stored, and/or shipped in advance (e.g., at a first time) while the BFS vial 210 that is pre-filled with the fluid may be manufactured, stored, and/or shipped at a later time (e.g., a second time). In some embodiments, the delay between the first time and the second time may be lengthy without causing determinantal effects, as the administration component 230 may be stored, in some embodiments, indefinitely. In such a manner, units of the administration component 230 may be provided to be on-hand in advance of the availability and/or arrival of the BFS vial 210, reducing supply chain constraints in the case of proactive administration component 230 procurement.

According to some embodiments, the components 210, 230 may be coupled, e.g., in the field and/or in situ, to provide an active pre-filled (e.g., injectable) medical delivery device. As shown in FIG. 2B, for example, the seal 234 may be removed from the administration component 230 (and/or the shield base 292; at “A”) and the administration component 230 (and/or the socket 232-1 and/or the shield base 292 thereof) may be aligned with the neck 212 of the BFS vial 210. According to some embodiments, the administration component 230 (and/or the mounting collar 232 and/or the shield base 292 thereof) may be axially engaged to couple with the BFS vial 210 via application of a mating axial force, as shown in FIG. 2C (at “B”). The administration component 230 (and/or the mounting collar 232 and/or the shield base 292 thereof) may be urged onto the neck 212 of the BFS vial 210, for example, such that the cooperatively shaped internal seat 238 (e.g., an interior radial groove or channel) accepts the mounting flange 216, thereby removably coupling the BFS vial 210 and the administration component 230 (and/or the mounting collar 232 and/or the shield base 292 thereof). In some embodiments, the internal seat 238 (and/or other interior features) and/or the mounting flange 216 may be shaped such that uncoupling of the BFS vial 210 and the administration component 230 (and/or the mounting collar 232 and/or the shield base 292 thereof) is mechanically prohibited. In some embodiments, the mounting flange 216 may be shaped as an axially elongated rounded exterior flange (e.g., the “doughnut” shape as depicted) and/or the internal seat 238 may comprise a cooperative and/or mirrored axially elongated rounded interior groove or track. According to some embodiments, the one or more mounting features 236 such as the interior radially-spaced axial grooves depicted may engage with the bottle flange 218 (and/or portions of the BFS vial 210) in a case where the safety shield 290 is not utilized, such that rotation of the administration component 230 (and/or the mounting collar 232 thereof) with respect to the BFS vial 210 is restricted in the case that they are coupled. In some embodiments, the one or more mounting features 236 such as the interior radially-spaced axial grooves depicted may engage with the mounting features 294 of the safety shield 290 (and/or portions of the shield base 292) in a case where the safety shield 290 is utilized and is disposed between the BFS vial 210 and the administration component 230, such that rotation of the administration component 230 (and/or the mounting collar 232 thereof) with respect to the BFS vial 210 is not restricted in the case that they are coupled. In some embodiments, such as in the case that the mounting features 294 of the safety shield 290 engage with, are seated in, and/or are retained by corresponding mounting features 236 of the mounting collar 232, axial rotation of the mounting collar 232 with respect to the safety shield 290 may be mechanically prohibited. In some embodiments, the mounting features 296 of the safety shield 290 may not be utilized (or may comprise one or more continuous and/or non-rotation limiting features) and free rotation between the mounting collar 232 and the safety shield 290 may be mechanically permitted. According to some embodiments, the coupling of the BFS vial 210 and the administration component 230 (and/or the mounting collar 232 and/or the safety shield 290 thereof) may be configured to explicitly permit free rotation (e.g., about a common axis) of the BFS vial 210 with respect to the administration component 230 (and/or the mounting collar 232 and/or the safety shield 290 thereof). The mounting features 236 may not, in some embodiments for example, engage with the BFS vial 210 (and/or the mounting flange 216 thereof), e.g., to permit rotation therebetween.

As depicted in FIG. 2D, in some embodiments the neck 212 of the BFS vial 230 may be urged and/or forced into the socket 232-1 of the mounting collar 232 (and/or through the shield base 292, e.g., which may be annular in shape as depicted) until the mounting flange 216 becomes seated in (and/or coupled to or mated with) the internal seat 238 (e.g., a seated position). In such a manner, the fluid seal 214 may be advantageously positioned adjacent to the puncture seal 240. According to some embodiments, the mounting flange 216 may be configured as the doughnut shape (as depicted) to provide various advantages to the pre-filled medical delivery assembly 200. The axial elongation of the mounting flange 216 may, for example, provide for a smooth, uniform, and/or less forceful mating process that is less likely to deform the soft plastic neck 212 of the BFS vial 210 and/or may provide for a lengthened mating surface that is more likely to prevent leakage of the fluid. In some embodiments, the mounting flange 216 and the cooperatively shaped and sized internal seat 238 may permit simple, effective, and/or economic attachment of the administration component 230 to the BFS vial 210. According to some embodiments, e.g., in preparation for administration and/or utilization of the pre-filled medical delivery assembly 200, the shield element 296 of the safety shield 290 may be rotated, flipped, and/or otherwise repositioned via activation of the hinge element 292-1 (at “D”).

In some embodiments, the needle hub 250 may be coupled to the mounting collar 232 via engagement of the internal threads 242 of the mounting collar 232 with the external threads 252 (or thread) of the needle hub 250. The external threads 252 that correspond and cooperate with the internal threads 242 such that they may be rotationally and/or removably coupled. According to some embodiments, the administration component 230 may be provided with the mounting collar 232 and the needle hub 250 partially engaged (e.g., with the threads 242, 252 being partially coupled), such as depicted in FIG. 2D. In some embodiments, the partial engagement (or first engagement state) may cause the administration member 270 (e.g., a second or proximal end thereof) to be positioned adjacent to (and/or in contact with) the puncture seal 240 of the mounting collar 232. In such a first engagement state, the administration component 230 may be coupled to the BFS vial 210, but the BFS vial 210 (and/or the puncture seal 240) have not yet been punctured and/or breached by the administration member 270. In other words, the pre-filled medical delivery assembly 200 in FIG. 2A, FIG. 2B, FIG. 2C, and FIG. 2D is in a non-activated or pre-activated state.

According to some embodiments the needle hub 250 may couple to and/or retain the administration member 270. The administration member 270 may be inserted into and/or through the needle hub 250 (and/or the riser 256 thereof), for example, such that it comprises a first or administration end 272 extending axially distal from the BFS vial 210 and a second or piercing end 274 disposed within the needle hub 250. In some embodiments, the administration end 272 and/or a distal portion of the administration member 270 may be housed, shrouded, and/or covered by the cap 280. According to some embodiments, the cap 280 may be configured to house the administration member 270 and to removably couple to the housing 250 (e.g., by fitting over an external portion thereof and/or by engaging with the external flange 244).

According to some embodiments, the mounting collar 232 and needle hub 250 (and cap 280) combination may be utilized to couple and/or mate the administration member 270 with the BFS vial 210 to provide a mechanism via which the administration member 270 may be coupled to the soft plastic BFS vial 210 in a reliable manner. Due to the nature of the BFS plastic and/or process and/or the small form-factor of the BFS vial 210, for example, providing standard external threads (not shown) directly on the neck 212 would not be a viable option for it would result in an imprecise, unreliable, and/or non-water tight coupling (i.e., the threads would be deformable even if they could be properly manufactured to within the desired tolerances, which itself is not a likely result) between he BFS vial 210 and, e.g., the needle hub 250. Applicant has realized, for example, that “soft” plastics required for the BFS process are not susceptible to machining due to heat deformation of machined features during formation attempts as well as deformation due to mechanical stress during utilization. As such, standardized screw-on needle hubs (not shown) are not readily compatible for direct attachment to BFS vials 210 (e.g., in the absence of the mounting collar 232).

In some embodiments, the administration member 270 may include a needle shaped and/or sized for at least one of subcutaneous, intramuscular, intradermal, and intravenous injection of the fluid agent into the patient. For ease of explanation and description, the figures and the description herein generally refer to the administration member 270 as a needle or cannula. However, it should be noted that, in other embodiments, the administration member 270 (e.g., the administration end 272 thereof) may include a nozzle (not shown) configured to control administration of the fluid agent to the patient. The nozzle may include a spray nozzle, for example, configured to facilitate dispersion of the fluid agent into a spray. Accordingly, a needle hub 250 fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a needle hub 250 including a droplet nozzle may be useful in the administration of a fluid agent by way of droplets, such as administration to the eyes, topical administration, and the like.

As generally understood, the fluid or drug (e.g., stored in the BFS vial 210 and/or one or more of the reservoirs 220, 222 thereof) agent may include any type of agent to be injected into a patient (e.g., animal such as a mammal, either human or non-human) and capable of producing an effect (alone, or in combination with an active ingredient). Accordingly, the agent may include, but is not limited to, a vaccine, a drug, a therapeutic agent, a medicament, a diluent, and/or the like. According to some embodiment, either or both of the fluid agent and the active ingredient (i.e., the drug agent and/or components thereof) may be tracked, monitored, checked for compatibility with each other, etc., such as by utilization of electronic data storage devices (not shown) coupled to the various modules or components such as the BFS vial 210 (e.g., at, on, or in the identification area 224) and/or the administration component 230.

According to some embodiments, the mounting collar 232, the needle hub 250, and/or the cap 280 may be composed of a medical grade material. In some embodiments, the mounting collar 232, the needle hub 250, and/or the cap 280, may be composed of a thermoplastic polymer or other “hard” plastic (e.g., greater than 80 on the Rockwell “R” scale), including, but not limited to, polybenzimidazole, ABS, polystyrene, polyvinyl chloride, or the like. In some embodiments, the pre-filled medical delivery assembly 200 may be advantageously manufactured (in mass quantities) in separate parts or portions, namely, at least the “soft” plastic BFS vial 210 portion (e.g., a “first” piece) and the “hard” plastic administration component 230 (e.g., the “second” piece), with such different plastic parts/portions being selectively coupled to administer a medication to a patient.

According to some embodiments, the pre-filled medical delivery assembly 200 may be advanced from the first engagement state (i.e., the non-activated state) to a second engagement (or activation) state where the administration member 270 has pierced the BFS vial 210 and the fluid therein may readily be expressed through the administration member 270 (e.g., and into a patient). The partial engagement of the mounting collar 232 and the needle hub 350 (e.g., with the threads 242, 252 being partially coupled) as depicted in FIG. 2D may, for example, be transitioned to a more advanced, fully, or completely advanced state by application of a rotational force to the cap 280, e.g., as shown in FIG. 2D (at “C”). In some embodiments, the more advanced and/or full engagement (or second engagement state; as shown in FIG. 2E) may cause the administration member 270 (e.g., the second or proximal end 274 thereof) to advance through the puncture seal 240 of the mounting collar 232 and through the seal 214 of the BFS vial 210. In such a manner, for example, the continued rotational engagement of the threads 242, 252 may cause the administration member 270 (e.g., the second or proximal end 274 thereof) to open a fluid pathway between the BFS vial 210 (e.g., and/or the neck 212, and/or reservoirs 220, 222 thereof) and the first or distal end 272 of the administration member 270. In some embodiments, the cap 280 may be utilized as a rotational force driver to transfer rotational force to the needle hub 250 to cause the advancement of the engagement of the threads 242, 252 (and, e.g., the puncturing of the puncture seal 240 and the seal 214 of the BFS vial 210). The inside of the cap 280 may comprise a driver element (not shown), for example, that is sized and/or shaped to engage with the drive surface 254-1 of the needle hub 250 such that rotational force applied to the cap 280 is transferred to and rotationally advances the needle hub 250. In such a manner, for example, the cap 280 may be utilized to complete the threading/mating of the threads 242, 252 and thereby causing the second or proximal end 274 of the administration member 270 to pierce each of the puncture seal 240 and the seal 214 of the BFS vial 210. According to some embodiments, one or more of the reservoirs 220, 222 may be compressed (e.g., via application of radially inward force) to expel the fluid therein through the administration member 270. In some embodiments, once the administration is complete, the shield element 296 of the safety shield 290 may be repositioned (e.g., via activation and/or utilization of the hinge element 292-1) such as by flipping the shield element 296 (at “E” in FIG. 2E) to cover the administration member 270—e.g., as depicted in FIG. 2F.

According to some embodiments, the second or piercing end 274 of the administration member 270 may comprise a multi-bevel tip as described herein. The multi-bevel tip may, for example, decrease the likelihood of coring of either the puncture seal 240 and the seal 214, thereby reducing the likelihood of contamination of the fluid agent and/or blockage of the administration member 270. While standard tips may function adequately in the case of a linear axial piercing motion (not shown herein), for example, the rotational axial advancement of the second or piercing end 274 of the administration member 270 in embodiments described herein may produce undesirable results in the case that a standard or simple tip is utilized on the second or piercing end 274 of the administration member 270. In some embodiments, the first or distal end 272 of the administration member 270 may comprise any type or configuration of tip that is or becomes known or practicable for engagement with an administration target (e.g., a patient). According to some embodiments, the tip of the first or distal end 272 of the administration member 270 may comprise a multi-bevel tip similar to the second or piercing end 274 of the administration member 270 (e.g., such that during manufacturing and/or assembly of the administration component 230 the administration member 270 may be oriented in either longitudinal manner without change in effect).

In some embodiments, fewer or more components 210, 212, 214, 216, 218, 220, 222, 224, 230, 232, 232-1, 234, 236, 238, 240, 242, 244, 250, 252, 254-1, 256, 270, 272, 274, 280, 290, 292, 292-1, 294, 296, 298, 298-1 and/or various configurations of the depicted components 210, 212, 214, 216, 218, 220, 222, 224, 230, 232, 232-1, 234, 236, 238, 240, 242, 244, 250, 252, 254-1, 256, 270, 272, 274, 280, 290, 292, 292-1, 294, 296, 298, 298-1 may be included in the pre-filled medical delivery assembly 200 without deviating from the scope of embodiments described herein. In some embodiments, the components 210, 212, 214, 216, 218, 220, 222, 224, 230, 232, 232-1, 234, 236, 238, 240, 242, 244, 250, 252, 254-1, 256, 270, 272, 274, 280, 290, 292, 292-1, 294, 296, 298, 298-1 may be similar in configuration and/or functionality to similarly named and/or numbered components as described herein. According to some embodiments, the pre-filled medical delivery assembly 200 may comprise the mounting flange 216 but not the collapsible reservoir 220. In some embodiments, the pre-filled medical delivery assembly 200 may comprise the mounting flange 216 but not the dispensing reservoir 222. According to some embodiments, the administration component 230 may be provided without (and/or separately from) the BFS vial 210. In some embodiments, the safety shield 290 may not be included and/or utilized. According to some embodiments, the puncture seal 240 may instead comprise an opening or orifice or may not be included in the mounting collar 232. In some embodiments, the administration member 270 may be provided separate from the BFS vial 210 and/or the administration component 230.

### IV. Multi-Bevel Cannula Apparatus and Systems

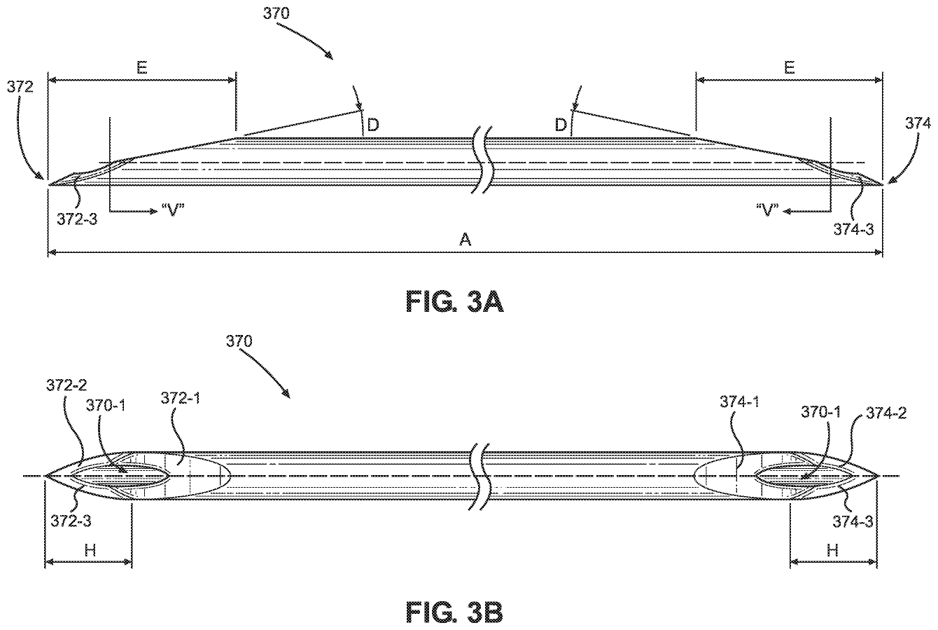

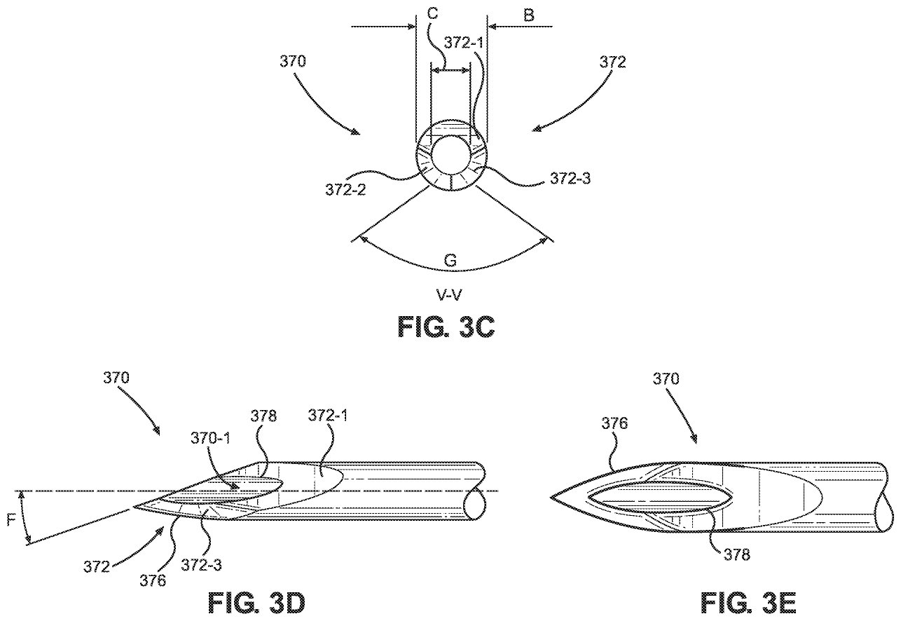

Referring now to FIG. 3A, FIG. 3B, FIG. 3C, FIG. 3D, and FIG. 3E, various views of a multi-bevel cannula 370 (e.g., apparatus) according to some embodiments are shown. The multi-bevel cannula 370 is not depicted to scale, for ease of illustration. In some embodiments, the multi-bevel cannula 370 may generally comprise a hollow tube defining an interior bore 370-1, having a length “A” (FIG. 3A), an outer diameter “B” (FIG. 3C), and an inner diameter “C” (FIG. 3C). In some embodiments, the multi-bevel cannula 370 may comprise a twenty-three gauge (23 g) tube. The length “A” of the multi-bevel cannula 370 may extend between a first tip or end 372 and a second tip or end 374. According to some embodiments, the ends 372, 374 may both be sharpened, ground, buffed, polished, and/or otherwise shaped to form points. As depicted, for example, the first end 372 may comprise a first primary bevel 372-1 and/or the second end 374 may comprise a second primary bevel 374-1, either or both being defined by cutting, grinding, or otherwise shaping the respective ends 372, 374 along a first plane disposed at a first angle “D” (FIG. 3A) with respect to a central longitudinal axis (and/or side-wall longitudinal axis) of the multi-bevel cannula 370. The primary bevels 372-1, 374-1 may extend longitudinally inward from each respective end 372, 374 by a primary bevel length “E” (FIG. 3A).

In some embodiments, the length “A” may be between thirty-two millimeters (32-mm) and fifty-four millimeters (54-mm) with a variance or tolerance of five tenths of a millimeter (0.5-mm). According to some embodiments, the outer diameter “B” may be between six tenths of a millimeter (0.6-mm) and six hundred and seventy-three thousandths of a millimeter (0.673-mm), e.g., a nominal diameter of six hundred and forty-two millimeters (0.642-mm) which is standard for a twenty-three gauge (23 g) cannula. In some embodiments, the inner diameter “C” may be between thirty-seven hundredths of a millimeter (0.37-mm) and four hundred and fifty-nine thousandths of a millimeter (0.459-mm). According to some embodiments, the first angle “D” may comprise eleven degrees (11°) with a variance or tolerance of one degree (1°) and/or the primary bevel length “E” may be two and eight tenths millimeters (2.8-mm) with a variance or tolerance of two tenths of a millimeter (0.2-mm).

According to some embodiments, the first end 372 may comprise a first secondary bevel 372-2 and/or the second end 374 may comprise a second secondary bevel 374-2, either or both being defined by cutting, grinding, or otherwise shaping the respective ends 372, 374 along a second plane disposed at a second angle “F” (FIG. 3D) with respect to the central longitudinal axis (and/or side-wall longitudinal axis) of the multi-bevel cannula 370. In some embodiments, the first end 372 may comprise a first tertiary bevel 372-3 and/or the second end 374 may comprise a second tertiary bevel 374-3, either or both being defined by cutting, grinding, or otherwise shaping the respective ends 372, 374 along a third plane disposed at the second angle “F” (FIG. 3D) with respect to the central longitudinal axis (and/or side-wall longitudinal axis) of the multi-bevel cannula 370. According to some embodiments, the second angle “F” may comprise eighteen degrees (18°) and/or may be otherwise greater than the first angle “D”. In some embodiments, the second and third planes may be angled with respect to the first plane. As depicted in FIG. 3C, for example, the second and third planes may define a rotation angle “G” which may be between one hundred degrees (100°) and one hundred and twenty degrees (120°), according to some embodiments. The rotation angle “G” may represent the rotational range imparted to the multi-bevel cannula 370 during manufacture after the primary bevels 372-1, 374-1 have been created, to symmetrically offset the formation of the secondary bevels 372-2, 374-2 and tertiary bevels 372-3, 374-3 with respect to the primary bevels 372-1, 374-1. According to some embodiments, the secondary bevels 372-2, 374-2 and/or the tertiary bevels 372-3, 374-3 may comprise lengths “H” that are between forty-five percent (45%) and fifty-five percent (55%) of the primary bevel length “E”.

In some embodiments, at least one of the ends 372, 374 is formed as a triple-bevel point comprising each of the respective primary bevels 372-1, 374-1, secondary bevels 372-2, 374-2, and tertiary bevels 372-3, 374-3. In such a manner, for example, rotational axial engagement of the at least one end 372, 374 with a BFS vial (not shown) may be performed with a reduced likelihood of coring. According to some embodiments, the multi-bevel cannula 370 may also or alternatively be honed or otherwise treated to reduce the likelihood of coring. Formation of the primary bevels 372-1, 374-1 creates or defines, for example, both an exterior edge 376 and an interior edge 378. Additional formation of the secondary bevels 372-2, 374-2 and/or tertiary bevels 372-3, 374-3 further defines and/or shapes each of the exterior edge 376 and the interior edge 378. In accordance with standard manufacturing processes, a product such as the multi-bevel cannula 370 would likely be honed, ground, sanded, polished, buffed, and/or otherwise treated to remove any burrs (not shown) or imperfections from the primary bevels 372-1, 374-1, secondary bevels 372-2, 374-2, tertiary bevels 372-3, 374-3, exterior edge 376, and interior edge 378. In accordance with some embodiments, the interior edge 378 may be additionally honed and/or treated (e.g., ground, sanded, polished, and/or buffed) beyond an extent normally required for simple burr removal and to a greater extent that causes the interior edge 378 to be dulled. The interior edge 378 may, in some embodiments, be dulled to a Brubacher Edge Sharpness Scale (BESS) rating of higher than five hundred (500), an edge apex thickness of greater than one micrometer (1 micron), and/or have a Cutlery and Allied Trades Research Association (CATRA) Razor Edge Sharpness Test (REST) push-cutting force of greater than five Newtons (5 N). According to some embodiments, the interior edge 378 may be dulled to a BESS rating of above six hundred (600), e.g., a BESS rating of one thousand (1000). In some embodiments, such dulling and/or honing may be conducted utilizing glass bead blasting/honing for a duration in excess of that which is typical for burr removal and for a duration that is measured to produce a dull edge for the interior edge 378. According to some embodiments, the dull interior edge 378 may reduce the likelihood of and/or substantially prevent coring when engaged with a soft plastic BFS vial in a rotational axial piercing manner (e.g., as utilized for activation of each of the pre-filled medical delivery assemblies 100, 200 of FIG. 1A, FIG. 1B, FIG. 1C, FIG. 1D, FIG. 1E, FIG. 1F, FIG. 2A, FIG. 2B, FIG. 2C, FIG. 2D, FIG. 2E, FIG. 2F, and FIG. 2G herein). While a sharpened exterior edge 376 may engage and pierce a BFS seal, for example, the interior edge 378 is unlikely (when dulled) to separately cut and/or core the BFS seal, resulting in a piercing without coring. According to some embodiments, the triple-bevel of a piercing end 372, 374 of the multi-bevel cannula 370 and/or the dulling of the interior edge 378 may also or alternatively provide for piercing of a BFS vial seal without splitting a seam (not shown) of the BFS vial. In the case that the multi-bevel cannula 370 pierces a BFS vial at a seam junction, for example, the embodiments presented herein may reduce the likelihood that such seam will split (e.g., causing leakage and/or failure), as opposed to being pierced as desired.

In some embodiments, fewer or more components 370-1, 372, 372-1, 372-2, 372-3, 374, 374-1, 374-2, 374-3, 376, 378 and/or various configurations of the depicted components 370-1, 372, 372-1, 372-2, 372-3, 374, 374-1, 374-2, 374-3, 376, 378 may be included in the multi-bevel cannula 370 without deviating from the scope of embodiments described herein. In some embodiments, the components 370-1, 372, 372-1, 372-2, 372-3, 374, 374-1, 374-2, 374-3, 376, 378 may be similar in configuration and/or functionality to similarly named and/or numbered components as described herein. According to some embodiments, only one of the ends 372, 374 may comprise a triple-bevel point. In some embodiments, one or more of the ends 372, 374 may comprise a dual-bevel point comprising both of a respective primary bevel 372-1, 374-1 and secondary bevel 372-2, 374-2.

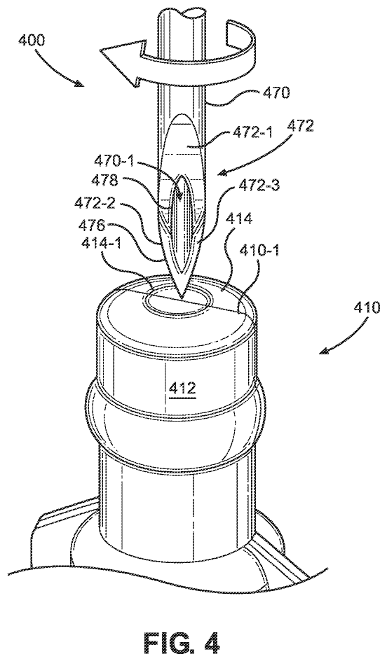

Turning finally to FIG. 4, a partial perspective view of a multi-bevel cannula pre-filled medical delivery system 400 according to some embodiments is shown. In some embodiments, the multi-bevel cannula pre-filled medical delivery system 400 may comprise a BFS vial 410 comprising a seam 410-1 and/or defining a neck 412 that terminates at a seal 414. According to some embodiments, the seal 414 may comprise and/or define a piercing region 414-1 that, e.g., may comprise a thinner or thicker walled portion of the BFS vial 410 that is centered on the circular end of the neck 412. In some embodiments, the multi-bevel cannula pre-filled medical delivery system 400 may comprise a cannula 470 (e.g., a cylindrical tube defining an interior passage 470-1) disposed (e.g., coupled and/or positioned; although coupling and/or positioning structure is not shown in FIG. 4 for ease of illustration) axially in-line with the BFS vial 410 and comprising a tip 472 positioned adjacent to the seal 414. According to some embodiments, the cannula 470 (e.g., the tip 472 thereof) may comprise a triple-bevel defined by a first bevel surface 472-1, a second bevel surface 472-2, and a third bevel surface 472-3. In some embodiments, the triple-bevel may define an external edge 476 and an internal edge 478. According to some embodiments, the internal edge 478 may be dulled, e.g., to reduce a likelihood of coring of the seal 414.

In some embodiments, the triple-bevel cannula 470 (e.g., with dulled internal edge 478) may be advanced to pierce the seal 414. According to some embodiments, the advancement may comprise both axial and rotational motion (e.g., in accordance with the methods of piercing defined by the structural components of the pre-filled medical delivery assemblies 100, 200 of FIG. 1A, FIG. 1B, FIG. 10, FIG. 1D, FIG. 1E, FIG. 1F, FIG. 2A, FIG. 2B, FIG. 2C, FIG. 2D, FIG. 2E, FIG. 2F, and FIG. 2G herein). In the case that the cannula 470 is coupled to a hub or driving member (not shown) that engages via threads to a connector (also not shown in FIG. 4) coupled to the BFS vial 410, for example, engagement of the threads may advance the point 472 proximate to the BFS vial 410 towards the seal 414 thereof (e.g., including a rotational motion vector). In some embodiments, the multi-bevel point 472 may alter the engagement of the cannula 470 with the BFS vial 410 such that undesirable effects are minimized or entirely avoided. Incorporation of the multi-bevel (e.g., triple-bevel) point 472 for engagement of the BFS vial 410 may, for example, provide for a more uniform and/or predictable piercing of the seal 414 while reducing or eliminating undesirable coring effects. It has been discovered, for example, that a simple bevel pointed cannula (not shown) advanced in a rotational manner into a BFS vial 410 seal 414 may cause undesirable tears, splitting, fracturing, splintering, coring, and/or debris.

In some embodiments, fewer or more components 410, 410-1, 412, 414, 414-1, 470, 472, 472-1, 472-2, 472-3, 476, 478 and/or various configurations of the depicted components 410, 410-1, 412, 414, 414-1, 470, 472, 472-1, 472-2, 472-3, 476, 478 may be included in the multi-bevel cannula pre-filled medical delivery system 400 without deviating from the scope of embodiments described herein. In some embodiments, the components 410, 410-1, 412, 414, 414-1, 470, 472, 472-1, 472-2, 472-3, 476, 478 may be similar in configuration and/or functionality to similarly named and/or numbered components as described herein.

### V. Methods for Rotational Piercing of Pre-Filled Medical Delivery Devices

In some embodiments, various methods and/or processes may be performed and/or implemented to utilize a BFS vial and/or bottle filled with a single dose of medicament to administer the single dose to a patient/target. In some embodiments, a method may cause a BFS vial to be coupled to an administration component that is engaged to puncture the BFS vial (e.g., in a rotational axial manner and/or utilizing a multi-bevel cannula as described herein) and then may be utilized to inject (or otherwise administer) the medicament to the patient/target. The methods and/or processes (and/or diagrams and/or flow diagrams thereof) described herein do not necessarily imply a fixed order to any depicted actions, steps, and/or procedures, and embodiments may generally be performed in any order that is practicable unless otherwise and specifically noted. While the order of actions, steps, and/or procedures described herein is generally not fixed, in some embodiments, actions, steps, and/or procedures may be specifically performed in the order listed, depicted, and/or described and/or may be performed in response to any previously listed, depicted, and/or described action, step, and/or procedure.

In practice, for example, some or all of the following procedures may be followed to utilize a pre-filled medical delivery assembly to administer a medication (and/or other fluid) to a patient/target. In some embodiments, the area of injection may be cleaned and/or otherwise prepared. A neck and/or a fluid seal of the BFS vial (e.g., a “first” part and/or component) may be cleaned (e.g., utilizing an alcohol wipe) to prepare the BFS vial for coupling to an administration component and/or assembly. In some embodiments, a “second” part and/or component comprising a pre-packaged mounting collar/coupling, needle hub (with an administration member such as a multi-bevel cannula), cap, and/or safety shield may comprise a seal that maintains an internal volume/fluid passage in a sterile state and the seal may be removed to repaper for the coupling to the BFS vial. According to some embodiments, the administration assembly may be axially aligned with the neck of the BFS vial and the neck may be inserted into the mounting collar and/or through a base member of the safety shield to engage a mounting flange with a cooperatively shaped interior mating feature. According to some embodiments, the administration component (and/or the mounting collar and/or the safety shield thereof) may “click” or snap onto the BFS vial—e.g., thereby achieving an assembled but pre-activated or non-activated state (e.g., the BFS vial has not yet been punctured).

According to some embodiments, the needle hub may, as a component of a pre-packaged “second” part/administration assembly for example, be only partially engaged with (e.g., partially threaded onto) the mounting collar. Only a portion of the threads may be engaged, for example, such that the “second” part is at least loosely or partially coupled as a single object/assembly but the administration member is not advanced axially enough to pierce the BFS vial in the case that the “second” part/assembly is mated with the BFS vial. In such a manner, for example, a user may couple the “first” and “second” parts and then selectively engage the administration member to puncture the BFS vial (e.g., by application of rotational force to advance the threads/mating thereof) at some time later. According to some embodiments, a seal or wrapper may be placed around or across the mating line of the mounting collar and the needle hub, thereby maintain the level of thread advancement at a desired (e.g., partially-engaged) level until the seal/wrapper is removed by a user.

In some embodiments (once the seal/wrapper covering the threads is removed, in the case that one is utilized), a user may hold the mounting collar with one hand/fingers and thread (e.g., continue threading) the needle hub fully into the mounting collar by applying rotational force to the cap. The cap may comprise an internal key that engages with the one or more stop and/or drive features of the needle hub, for example, to transfer the rotational force to the needle hub and accordingly advance the mating of the threads. As the threading/mating advances, a piercing end of the administration member may be axially advanced (e.g., while rotating) to pierce through the mounting collar (e.g., a seal portion thereof) and/or through the fluid seal of the BFS vial, thereby activating the pre-filled medical delivery assembly. According to some embodiments, the piercing end of the administration member may comprise a multi-bevel cannula and/or may comprise a dulled interior edge as described herein, e.g., to reduce a likelihood of or prevent coring and/or other negative effects of the piercing operation. In some embodiments, the stop/drive features may be shaped to comprise one or more surfaces that engage with the internal key of the cap in the case that the cap is rotated in a clockwise direction but may comprise an opposing surface or feature that permits (and/or forces) the internal key to disengage with the stop/drive features in the case that the cap is rotated in a counter-clockwise direction. In such a manner, for example, a user may readily couple (or complete the coupling of) the needle hub and the mounting collar but may be prevented from utilizing the cap to unscrew or decouple the needle hub and the mounting collar. In some embodiments, the internal key may be configured to accept a threshold amount of torque before a designed failure so that the user may readily tighten the threads but any attempt to overtighten will result in separation or destruction of the internal key, thereby preventing the cap from further functioning as a driver of the mating process and accordingly preventing overtightening of the mating between the needle hub and the mounting collar. In some embodiments, an audible “click” or other sound of the failure of the internal key may comprise a designed indication to the user that the pre-filled medical delivery assembly is properly and/o fully assembled/activated.

According to some embodiments, the cap may be removed to reveal the administration member and/or an administration end thereof. In some embodiments, the administration member (e.g., the administration end thereof) may be inserted into the patient and a collapsible reservoir of the BFS vial may be squeezed (e.g., receive an application of radially inward force), thereby expelling the fluid through the administration member and into the patient. In some embodiments, the administration member may be withdrawn from the patient and/or a safety shield (e.g., coupled to and/or part of the administration component/assembly) may be selectively moved (e.g., flipped and/or rotated) into position to cover the administration member/needle. The pre-filled medical delivery assembly may then be properly disposed of. While the mounting collar and the needle hub are generally described and depicted as separate couplable objects, in some embodiments they may be manufactured (e.g., molded) as a single object or piece or may comprise additional pieces or parts.

In some embodiments, a multi-bevel needle may be formed by grinding or cutting a tube of material (e.g., steel) at a first angle at a first end thereof, thereby forming a primary bevel. The primary bevel may form or define an exterior edge as well as an interior edge, e.g., where the inner bore has been cut. According to some embodiments, the inner edge may be dulled, e.g., to prevent coring as described herein. In some embodiments, one or more additional or secondary bevels may be added such as by rotating the needle about a central axis by a certain offset amount (e.g., in a first rotational direction) and grinding or cutting a second bevel at a second angle. In some embodiments, the second angle may be steeper (e.g., greater) than the first angle. According to some embodiments, the needle may be rotated back to the original rotational position and then rotated in an opposite (e.g., second) rotational direction by the offset amount and then ground or cut to form a third bevel at the second angle (or a third angle). In such a manner, for example, two secondary and symmetrical bevels may be added to the primary bevel, producing a triple-bevel tip or end to the needle. According to some embodiments, a second end of the needle may be formed with a single, double, or triple-bevel, e.g., in a similar manner.

### VI. Rules of Interpretation

Throughout the description herein and unless otherwise specified, the following terms may include and/or encompass the example meanings provided. These terms and illustrative example meanings are provided to clarify the language selected to describe embodiments both in the specification and in the appended claims, and accordingly, are not intended to be generally limiting. While not generally limiting and while not limiting for all described embodiments, in some embodiments, the terms are specifically limited to the example definitions and/or examples provided. Other terms are defined throughout the present description.

As utilized herein, the term “Blow-Fill-Seal” or “BFS” is utilized to refer to the manufacturing process well-known in the industry for producing large quantities of pre-filled plastic containers in a sterile environment. While certain implementations utilize forced air to “blow” extruded resin/parasin into the molds, in some implementations vacuum force may also or alternatively be applied to, e.g., “suck” the extruded resin/parasin into the mold cavities.

As utilized herein, the terms “cannula” and “needle” are utilized interchangeably to generally refer to a thin, elongated tube that is operable to function as a small conduit through which a medicament may be passed to treat a patient/target. In embodiments where a cannula/needle comprises a point or tip (e.g., due to formation of at least one bevel to at least one end thereof), the delivery of the medicament may be via means of injection into a patient/target. A beveled canula/needle that is coupled to a BFS container generally defines an injectable BFS device or system.

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention(s) may be practiced with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

Devices that are in communication with each other need not be in continuous communication with each other, unless expressly specified otherwise.

A description of an embodiment with several components or features does not imply that all or even any of such components and/or features are required. On the contrary, a variety of optional components are described to illustrate the wide variety of possible embodiments of the present invention(s). Unless otherwise specified explicitly, no component and/or feature is essential or required.

Further, although process steps, algorithms or the like may be described in a sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicants intend to file additional applications to pursue patents for subject matter that has been disclosed and enabled but not claimed in the present application.

It will be understood that various modifications can be made to the embodiments of the present disclosure herein without departing from the scope thereof. Therefore, the above description should not be construed as limiting the disclosure, but merely as embodiments thereof. Those skilled in the art will envision other modifications within the scope of the invention as defined by the claims appended hereto.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A pre-filled medical delivery assembly, comprising:
a blow-fill-seal (BFS) bottle defining a collapsible fluid chamber, a neck portion, and a rounded exterior flange formed on the neck portion;
a collar defining a first end and a second end, the first end defining a mounting socket comprising an interior seat into which the rounded exterior flange is axially mated and the second end defining a hub socket comprising internal threads;
a needle hub comprising at least one external thread cooperatively mated with the internal threads of the collar to a first degree of advancement, the needle hub being coupled to a double-ended needle disposed through the needle hub, the double-ended needle comprising a multi-bevel piercing tip; and
a cap covering an administration tip of the needle and the cap comprising an interior key that is operable to drive the needle hub to advance the at least one external thread of the needle hub to a second degree of advancement with respect to the internal threads of the hub socket of the collar,
wherein advancement of the threads to the second degree of advancement causes the multi-bevel piercing tip of the double-ended needle to pierce a seal of the BFS bottle.

2. The pre-filled medical delivery assembly of claim 1, further comprising a safety shield coupled to the BFS bottle.

3. The pre-filled medical delivery assembly of claim 2, wherein the safety shield comprises a shield base coupled to the BFS bottle and a hinge element joining the shield base to a shield element.

4. The pre-filled medical delivery assembly of claim 1, further comprising a safety shield coupled to the collar.

5. The pre-filled medical delivery assembly of claim 1, further comprising a safety shield comprising an annular shield base through which the BFS bottle neck portion is inserted.

6. The pre-filled medical delivery assembly of claim 5, wherein the shield base comprises one or more mounting features that axially couple to corresponding features of the collar.

7. The pre-filled medical delivery assembly of claim 1, wherein the interior seat comprises an interior channel rounded at a radius in the range of four to five millimeters (4-mm to 5-mm).

8. The pre-filled medical delivery assembly of claim 1, wherein the interior seat extends radially outward into an interior wall of the mounting socket by a protrusion amount and wherein a length of the interior seat is in the range of four and three tenths times (4.3×) and five and three tenths times (5.3×) the protrusion amount.

9. A medical administration assembly, comprising:
a collar defining a first end and a second end, the first end defining a mounting socket comprising an interior seat operable to accept an exterior flange of a blow-fill-seal (BFS) bottle that is axially mated and the second end defining a hub socket comprising internal threads;
a needle hub comprising at least one external thread cooperatively mated with the internal threads of the collar to a first degree of advancement, the needle hub being coupled to a double-ended needle disposed through the needle hub, the double-ended needle comprising a multi-bevel piercing tip; and
a cap covering an administration tip of the needle and the cap comprising an interior key that is operable to drive the needle hub to advance the at least one external thread of the needle hub to a second degree of advancement with respect to the internal threads of the hub socket of the collar,
wherein advancement of the threads to the second degree of advancement causes the multi-bevel piercing tip of the double-ended needle to pierce a seal of the collar.

10. The medical administration assembly of claim 9, wherein advancement of the threads to the second degree of advancement further causes the multi-bevel piercing end of the double-ended needle to pierce a seal of the BFS bottle.

11. The medical administration assembly of claim 9, further comprising a seal covering an opening of the mounting socket.

12. A multi-bevel cannula for a pre-filled medical delivery assembly, comprising:
an elongate hollow tube body portion defining an interior fluid conduit and a first end and a second end thereof;
the first end comprising a first bevel defining a first point for engagement with a patient; and
the second end comprise a triple-bevel defining a second point for rotational piercing of a BFS vial.

13. The multi-bevel cannula of claim 12, wherein the cannula is made of steel.

14. The multi-bevel cannula of claim 12, wherein the first bevel also comprises a triple-bevel.

15. The multi-bevel cannula of claim 12, wherein the cannula comprises a 23-gauge needle.

16. The multi-bevel cannula of claim 12, wherein the triple-bevel of the second point defines both an outside edge and an inside edge and wherein the inside edge is dulled.
